(19) United States
(12) Patent Application Publication
(10) Pub. No.: US 2015/0359975 A1
(43) Pub. Date: Dec. 17, 2015

(54) Injection needle and Method of Making Same

(71) Applicant: Anthony L. King, Oak Park, IL (US)

(72) Inventor: Anthony L. King, Oak Park, IL (US)

(21) Appl. No.: 14/735,733

(22) Filed: Jun. 10, 2015

Related U.S. Application Data
(60) Provisional application No. 62/011,170, filed on Jun. 12, 2014.

(51) Int. Cl. A61M 5/32; A61C 5/02; A61M 19/00

(52) U.S. Cl. CPC A61M 5/3286; A61M 19/00; A61C 5/026; A61M 2210/0631; A61M 2207/00

(57) ABSTRACT

An improved injection needle is made by swaging the proximal end of a standard gauge hypodermic needle. This results in both a smaller outside diameter, which lessens the initial injection pain, and a smaller inside diameter, which slows the flow of medication. The swaged proximal end also has a greater wall thickness than standard gauge needles with a larger inside diameter.

### RELATED APPLICATION

This application is based on, and claims the benefit of, U.S. Provisional Application No. 62/011,170 filed Jun. 12, 2014.

### BACKGROUND OF THE INVENTION

1. Field of the Invention

This invention relates generally to an improved dental anesthetic injection needle and method of making same for reducing patient pain upon injection of an anesthetic such as lidocaine.

2. Background Art

A dental syringe used by dentists for the injection of an anesthetic comprises a breech-loading syringe fitted with a sealed cartridge containing liquid anesthetic for delivery though a hollow needle injected into the patient's gum and mouth tissues. The most common, and perhaps most preferred, way of administering an anesthetic such as lidocaine prior to performing other dental procedures is with such a dental syringe grasped between the index and middle fingers of the dentist's one hand for insertion of the needle into the patient's gum and mouth tissues together with depression of the hypodermic plunger with the thumb of the same hand.

Generally, a topical anesthetic is applied prior to the insertion of the needle. Nevertheless, many patients experience pain from the administration of the injected anesthetic. Weldon U.S. Pat. No. 6,560,975 issued May 13, 2003 discloses limiting the depth of an injection into the mucosa only to the extent of penetration of an applied topical anesthetic for “pain-free” injections.

In addition to factors such as the bevel design of the needle tip and its sharpness, and lubricants such as a silicon coating, which can reduce the force of insertion and reduce trauma and pain from needle insertions, the size or gauge of an injection needle affects pain experienced during insertion. Needles having a larger outer diameter are more likely to cause pain than smaller diameter needles. Thus, for example, insertion of a 27 gauge needle has a greater chance of being reported as painful than insertion of a 30 gauge needle. The likelihood of bleeding also tends to decrease with decreasing needle diameter. Use of smaller guage, less painful needles, is limited by the need for small needles to reliably be inserted into gum and mouth tissues, to have sufficient mechanical strength, and to be manufactured in a cost-effective manner.

Conventionally, the accepted solution to increasing strength of a hollow tube is to go up a one tube size while retaining the original wall thickness for a proportionally greater increase in strength than weight. However, for injection needles that usually better solution is not workable as the lower gauge, increased outside diameter, needle not only is likely to result in greater risk of pain upon the initial injection, but would also increase the risk of administering the medication at too fast a rate because of the increased inside diameter.

Administration of an injected anesthetic requires not only getting the needle into the tissue, but also delivering the anesthetic. The force or rate at which the anesthetic is delivered is the principal reason for the patient's discomfort and pain. Currently 22 guage dental needles are often used and their internal diameter permits a maximum flow rate which can cause patient discomfort and pain if not properly administered. While it possible to reduce the flow rate by exercising control to reduce pressure on the dental syringe plunger, it requires patience and a delicate touch which are at times difficult to accomplish in the environment of an actual dental procedure.

Spinello U.S. Pat. No. 4,747,824 issued May 31, 1988 discloses effecting painless hypodermic needle administration of liquid anesthetics with a needle carried on a light wand and an electronic metered anesthetic delivery system. It can deliver the anesthetic very slowly through the needle means independent of the operator's manual exertion of pressure. However, start up and use per patient costs are high as compared to the standard type dental syringe. Moreover, it is necessary to change from the standard syringe hand position to a modified pen grasp resulting in a loss of tactile sensitivity. Such change of hand posture is a concern as the traditional landmarks and fulcrum that dentists use make it difficult to achieve with the pen grasp position.

There remains a need for a disposable injection needle useable with standard dental syringes that decreases the maximum flow rate of anesthetic via fluid mechanics.

### SUMMARY OF THE INVENTION

The present invention is concerned with providing an improved injection needle and method of making same for reducing patient pain upon injection of an anesthetic such as lidocaine. Such an improved injection needle has a distal end and a proximal end, with a distal portion extending from the distal end to an intermediate portion between the distal end and the proximal end, a proximal portion extending from the intermediate portion to the proximal end; and a generally cylindrical bore of a first inside diameter in the distal portion and a generally cylindrical bore of a second inside diameter in the proximal portion that is significantly smaller than the first inside diameter. The distal portion may be generally cylindrical and have a first outside diameter and the proximal portion may be generally cylindrical and have a second outside diameter that is significantly smaller than the first outside diameter. The distal portion may have a first wall thickness and the proximal portion may have a second wall thickness that is greater than the first wall thickness. The intermediate portion may be generally conical with the larger end adjacent the distal portion and the smaller end adjacent the proximal portion. The generally cylindrical bore of the distal portion and the generally cylindrical bore of the proximal portion may be generally co-axial, and the generally cylindrical distal portion and the generally cylindrical proximal portion may be generally co-axial.

The present invention is concerned with providing a method for making an improved injection needle for reducing patient pain upon injection of an anesthetic by swaging an injection needle to reduce the internal diameter of the proximal end. Additional steps may include bonding a plastic hub over the swaged joint area and grinding the bevel of the proximal end after swaging.

### BRIEF DESCRIPTION OF THE DRAWINGS

For a better understanding of the present invention, reference may be had to the accompanying drawings in which:

FIG. 1 is a showing of an existing, prior art device comprising a syringe, anesthetic cartridge, and needle in the hand of a dentist in preparation for injecting the anesthetic;

FIG. 2 is a perspective view of an improved injection needle of the present invention; and

FIG. 3 is an enlarged sectional view taken axially along the length of the improved injection needle view of the present invention shown in FIG. 2; and

FIG. 4 is an enlarged, distal end view of the improved injection needle of the present invention shown in FIG. 2.

### DETAILED DESCRIPTION

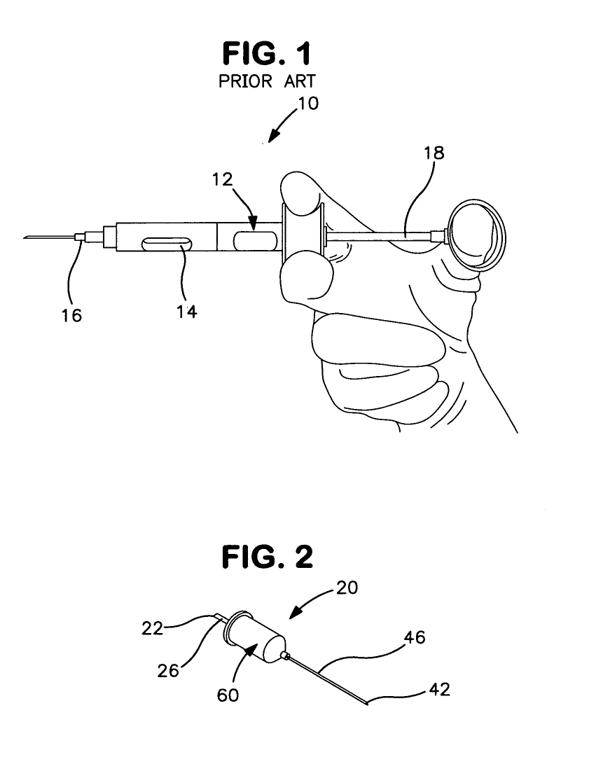

Referring now to the drawings, in which like parts are identified by like reference numerals throughout the several views, FIG. 1 illustrates an existing, prior art device 10 comprising a syringe 12, anesthetic cartridge 14, needle 16, and plunger 18 in the hand of a dentist in preparation for injecting the anesthetic into a patient prior to performing other dental work on the patient. Details of the type of prior art syringe illustrated in FIG. 1 vary, but generally accommodate grasping and activation by the dentist in a manner similar to that illustrated in FIG. 1, namely between the index and middle fingers of the dentist's one hand for insertion of the needle into the patient's gum and mouth tissues together with depression of the hypodermic plunger 18 with the thumb of the same hand. Needle 16 is a conventional prior art, generally cylindrical, hollow injection needle having substantially the same (within manufacturing tolerances) outside diameter throughout its length and having substantially the same (within manufacturing tolerances) inside diameter throughout its length. Syringe 12 is sterilized after each patient while anesthetic cartridge 14 and needle 16 are discarded after use and replaced with a new anesthetic cartridge and a new needle.

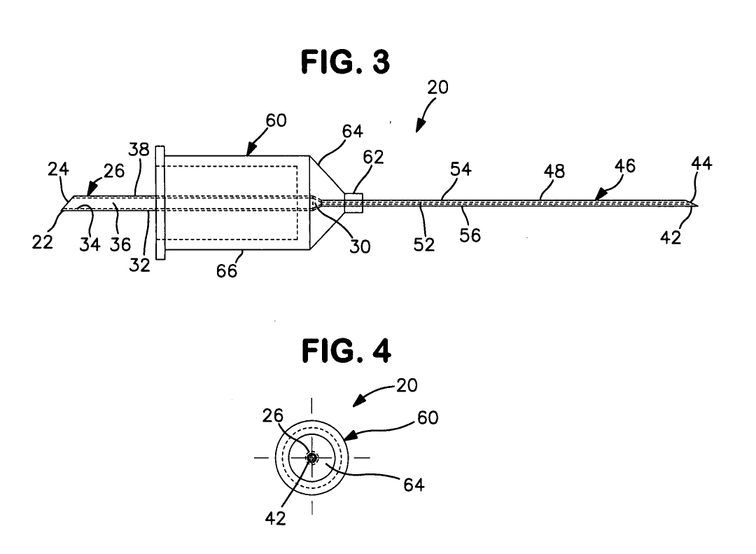

Shown in FIGS. 2 through 4 is an improved dental anesthetic injection needle 20 of the present invention for reducing patient pain upon injection of an anesthetic such as lidocaine. Needle 20, which replaces the prior art needle 16 in a syringe 12, has a distal end or perforator 22 with a sharp bevel 24, of any convenient conventional design, insertable into an anesthetic cartridge 14 through a diaphragm (not shown) carried by the cartridge. A distal portion 26 extends from end 22 to an intermediate, short truncated conical portion 30. Distal portion 26 is generally cylindrical and has a distal outer diameter 32 and a generally cylindrical bore 34 with a distal inner diameter 36 resulting in a distal wall thickness 38.

The opposite, proximal end or penetrator 42 with a sharp bevel 44, of any convenient conventional design, enters into the patient's tissue to deliver the anesthetic. A proximal portion 46 extends from intermediate, short truncated conical joint portion 30 to end 42. Proximal portion 46 is generally cylindrical and has a proximal outer diameter 48 and a generally cylindrical bore 52 with a proximal inner diameter 54 resulting in a proximal wall thickness 56.

As an example, needle 20 of the present invention may be made from a presently commercially available 22 gauge needle. Accordingly, distal end or perforator 22 would have an outer diameter 32 of approximately 0.72 mm and an inner diameter 36 of approximately 0.41 mm with a wall thickness 38 of approximately 0.15 mm. As illustrated in the drawings, distal portion 26 comprises about two-fifths of the distance from distal end 22. From about that distance, needle 20 is swaged creating intermediate short truncated conical joint section 30 and reducing both the outer diameter 48 and inner diameter 54 of the proximal portion 46 comprising the remaining approximately three-fifths of the length of needle 20. This process results in proximal portion 46 having an outer diameter 48 of approximately 0.41 mm and an inner diameter 54 of approximately 0.076 mm to 0.127 mm with a wall thickness 56 of approximately 0.16828 mm to 0.14288 mm.

Secured to needle 20 is a plastic hub 60 with a small proximal end 62 bonded to the reduced outer diameter of proximal portion 46. After swaging, either before or after bonding hub 60, bevel 44 of proximal end 42 is ground to restore it to proper sharpness. Extending back toward distal end 22, hub 60 has an intermediate truncated conical section 64 over swaged joint 30 and a larger hollow distal end 66 which receives anesthetic cartridge 14.

Using the same weight or pressure to depress the plunger 18 of a dental syringe 12 such as that illustrated in FIG. 1 results in a little more than double the time to deliver anesthetic through the above described needle of the present invention as compared to a standard 27 gauge needle and a corresponding decrease of the maximum flow rate by one-half. Administration of an anesthetic with a standard 1.8 cc syringe in less than one minute would be ideal for safe and reduced pain delivery. While such an ideal has not yet been achieved with the present invention, it is being closely approached.

Effectively, regardless of what the normal pressure of any clinician is, the time required for their delivery of an anesthetic into a patient's tissue is about doubled using needle 20 with an inner diameter even smaller than a standard 30 guage needle 16. In addition, while the outer diameter of the proximal portion of needle 20 entering the patient's tissue is essentially the same as that of a 27 guage needle 16, the wall thickness of that portion of needle 20 is significantly greater than that of a 27 guage needle 16 and more closely approximates the wall thickness of a 22 guage needle 16.

Accordingly, another advantage gained from the swaging process is an increase in the wall thickness of proximal end or penetrator 42 of needle 20, and hence its strength. Thus, the present invention results in an optimum combination of a smaller size, stronger penetrator 42 for reducing patient trauma upon injection and a reduced inner diameter for a slower and safer flow rate for delivery of the anesthetic.

A still further advantage of the present invention is that while flow of the anesthetic out of needle 20 is restricted, neither retraction nor back flow for aspiration is hindered or inhibited. Accordingly, the needle of the present invention, while slowing the delivery of anesthetic, still permits verification of the absence of blood or vascular cells from placement of the needle.

Solely as an example, the present invention has been particularly illustrated and described in the context of a dental anesthetic injection needle of a specific size.

While a particular embodiment of the invention has been shown and described, alternatives, variations and modifications will occur to those skilled in the art. It is intended in the appended claims to cover all such alternatives, variations and modifications that come within the true spirit and scope of the present invention.

What is claimed as new and desired to be secured by Letters Patent is:

1. An improved injection needle comprising:
a distal end and a proximal end penetrator;
a distal portion extending from the distal end to an intermediate portion between the distal end and the proximal end;
a proximal portion extending from the intermediate portion to the proximal end;
a generally cylindrical bore extending through from the distal end to the proximal end;
the generally cylindrical bore having a first inside diameter in the distal portion;
the generally generally cylindrical bore having a second inside diameter in the proximal portion; and
the second inside diameter being significantly smaller than the first inside diameter.

2. The improved injection needle of claim 1 further comprising:
the distal portion being generally cylindrical and having a first outside diameter;
the proximal portion being generally cylindrical and having a second outside diameter; and
the second outside diameter being significantly smaller than the first outside diameter.

3. The improved injection needle of claim 2 further comprising:
the distal portion having a first wall thickness;
the proximal portion having a second wall thickness; and
the second wall thickness being greater than the first wall thickness.

4. The improved injection needle of claim 3 in which the intermediate portion is generally conical with a larger end adjacent the distal portion and a smaller end adjacent the proximal portion.

5. The improved injection needle of claim 4 further comprising a hub having a forward end attached to the proximal portion.

6. The improved injection needle of claim 5 further comprising a hollow distal end in the hub for receiving at least part of an anesthetic cartridge in the hub opposite the forward end.

7. The improved injection needle of claim 6 in which the generally cylindrical bore in the distal portion and the generally cylindrical bore in the proximal portion are generally co-axial.

8. The improved injection needle of claim 7 in which the generally cylindrical distal portion and the generally cylindrical proximal portion are generally co-axial.

9. The improved injection needle of claim 1 further comprising:
the distal portion having a first wall thickness;
the proximal portion having a second wall thickness; and
the second wall thickness being greater than the first wall thickness.

10. The improved injection needle of claim 9 in which the intermediate portion is generally conical with a larger end adjacent the distal portion and a smaller end adjacent the proximal portion.

11. The improved injection needle of claim 10 further comprising a hub having a forward end attached to the proximal portion.

12. The improved injection needle of claim 11 further comprising a hollow distal end in the hub for receiving at least part of an anesthetic cartridge in the hub opposite the forward end.

13. The improved injection needle of claim 12 in which the generally cylindrical bore in the distal portion and the generally cylindrical bore in the proximal portion are generally co-axial.

14. The improved injection needle of claim 1 further comprising a hub having a forward end attached to the proximal portion.

15. The improved injection needle of claim 1 further comprising a hollow distal end in the hub for receiving at least part of an anesthetic cartridge in the hub opposite the forward end.

16. The improved injection needle of claim 15 in which the generally cylindrical bore in the distal portion and the generally cylindrical bore in the proximal portion are generally co-axial.

17. A method of making an improved injection needle having a distal end, a proximal end penetrator, and an opening extending from the distal end to the proximal end, comprising the step of swaging a portion of the injection needle to reduce the size of the opening adjacent the proximal end.

18. The method of making an improved injection needle of claim 17 further comprising the step of securing a hub over at least part of the swaged portion.

19. The method of making an improved injection needle of claim 17 further comprising the step of grinding a bevel on the proximal end after swaging.

20. The method of making an improved injection needle of claim 17 further comprising the steps of securing a hub over at least part of the swaged portion and grinding a bevel on the proximal end after swaging
